(19) United States
(12) Patent Application Publication
(10) Pub. No.: US 2009/0130622 A1
(43) Pub. Date: May 21, 2009

(54) Method and Apparatus for Disinfecting or Sterilizing a Root Canal System Using Lasers Targeting Water

(76) Inventors: James Edwin Bollinger, Westlake Village, CA (US); John David West, Fox Island, WA (US); Clifford J. Ruddle, Santa Barbara, CA (US)

Correspondence Address: WYATT, TARRANT & COMBS, LLP, 1715 AARON BRENNER DRIVE, SUITE 800, MEMPHIS, TN, 38120-4367, US

(21) Appl. No.: 12/271,341

(22) Filed: Nov. 14, 2008

Related U.S. Application Data
(60) Provisional application No. 60/988,651, filed on Nov. 16, 2007.
(60) Provisional application No. 61/035,945, filed on Mar. 12, 2008.

(51) Int. Cl. A61B 18/20; A61C 3/00

(52) U.S. Cl. 433/29; 606/13

(57) ABSTRACT

Method and apparatus for disinfecting and/or sterilizing a root canal system by targeting the water content of disease and debris in the canals. The laser technique of employs a frequency of the wavelength emissions between about 930 to about 1065 nanometers with an optimum of 980 nm. This range of wavelengths targets the water content of tissue cells and pathogens as well as any residual organic debris in water within the root canal system after its preparation while being poorly absorbed by the surrounding dentin. The selection of the optimum wavelength produces significant effects generating and advancing treatment to the targeted aqueous environments. This is due to the rapid energy absorption by the water and the subsequent creation of gas bubbles, liberation of heat and subsequent propulsion of waves of heat and gas that impact along the canal walls and ramifications resulting in an enhanced bacterial kill and cleaning of the canal walls and ramifications. No dyes or other additives are necessary to enhance the effectiveness of the laser kill of bacteria, etc.

### CROSS-REFERENCE TO RELATED APPLICATIONS

This application claims benefit of Provisional Application Ser. No. 60/988,651, filed Nov. 16, 2007 and Provisional Application Ser. No. 61/035,945, Filed Mar. 12, 2008, both entitled Method and Apparatus for Disinfecting or Sterilizing A Root Canal System Using Lasers Targeting Water, the full contents of which are incorporated herein, by reference.

### STATEMENT REGARDING FEDERALLY SPONSORED RESEARCH OR DEVELOPMENT

Not applicable

### REFERENCE TO A MICROFICHE APPENDIX

Not applicable

### FIELD OF THE INVENTION

The present invention relates to method and apparatus for endodontic laser procedures involving the sterilization and/or disinfection of root canal systems including the ablation, vaporization, killing, injury or removal of bacteria, viruses, yeasts, molds, fungi, biofilms and prions as well as the ablation/vaporization and/or removal of residual tissue and other intracanal debris.

### BACKGROUND OF THE INVENTION

This invention relates to a method and apparatus for disinfecting and/or sterilizing the internal root canal anatomy of a tooth and removing biofilms, tissue fragments, and other debris/toxins/substrates from all aspects of the root canal system, including the accessory anatomy as well as the apical and lateral external root surfaces through the selective use of laser light energy at a wavelength which is readily absorbed by water and water-bearing debris including bacteria, diseased tissue, and the like.

Within the interior of each tooth exists a system of channels and tunnels housing the dental pulp. This systems consist of larger primary canals (the primary system) and a system of smaller interconnected branches, fins, loops, webs, tributaries, cul-de-sacs, anastomoses and other smaller irregularities called the secondary anatomy or accessory anatomy (See FIGS. 10 and 11). The primary anatomy and the secondary anatomy, in combination, are referred to as the root canal system. No two root canal systems are alike and the exact morphology is never known to the clinician in advance of treatment. Accessory anatomy can occur anywhere along the length of the primary canal and in any form or combinations thereof.

Disease of the root canal system (endodontic or pulpal disease) involves degenerative changes of the dental pulp resulting in inflammatory changes or infection inside the root canal system. This disease process originates within the root canal system. Pulpal breakdown and disease flow frequently egresses along the anatomical pathways and gives rise to lesions of endodontic origin in the periodontal tissues. Such degenerative changes in the pulp can be brought about by cumulative or acute trauma. Such trauma may be indirect such as caries, occlusal loading, fractures, erosions, and restorative dentistry. In other instances, the etiology of pulpal degeneration is direct resulting from direct carious exposure of the pulp chamber or from acute trauma resulting from injuries that fracture the tooth crown and/or root exposing the pulp to frank invasion of the oral flora. Root canal infections are often mixed infections and may involve many types of micro-organisms, including bacteria, yeasts and some viruses. Since most of the infections are mixed infections and, primarily bacterial in nature, for simplicity's sake the term “infection”, as used herein, means the presence of multiple bacterial types such as, yeasts, viruses, prions, or any pathologic micro-organisms that inhabit the root canal space. The term “bacteria” is herein used in a similar broad, all inclusive, sense.

Regardless of the etiology of the infection, or the organisms involved, once the sterility of the root canal system is compromised, the pulp begins an irreversible course of degeneration, ultimately culminating in necrosis and complete infection of the root canal system and potentially the periradicular and periapical tissues.

Substrates left in the root canal system after treatment, such as residual tissue, blood, smear layer, etc., regardless of their source, serve to provide nourishment to these pathogens inhabiting the root canal space fostering their persistence, colonization, and multiplication. The infection first establishes itself within the root canal system and then inevitably exits the confines of the root canal system via any portal of exit to the root surface including iatrogenic and resorptive perforations. The egress of pathogenic irritants from the root canal space inside the tooth serve to infect the surrounding tissues exterior to the root of the tooth.

The root dentin surrounding the root canal system is comprised of between 80-120 thousand tubules per square millimeter. Thus, there is direct communication from the root canal space to the external root surface via the dentinal tubules. Such microtubules are difficult to clean chemomechanically during endodontic procedures. Bacteria in root canal infections deeply imbed themselves in these microtubules and become difficult to completely kill via established chemomechanical clinical protocols. It has been well established that virtually all micro-organisms will become dormant or die if the supply of nutrients or substrates is cut off. Therefore, it is essential that all tissue substrates be removed during the endodontic procedure.

The ultimate objective of clinical endodontic treatment is to eliminate all pulpal tissue, bacteria and their related irritants, from the root canal system. Failure to eliminate pathogens during endodontic treatment contributes to many treatment failures, retreatments, surgeries, and extractions. Current methods of disinfection in the treatment of root canal disease involve mechanically preparing or shaping canals and the attempted chemical disinfection of the primary and secondary anatomy.

It should be completely understood and fully appreciated that it is difficult to clean both the dentinal tubules and secondary anatomy in that, by definition, these complex micropores cannot be enlarged mechanically due to their extremely small size and the fact that the angle of access and the angle of incidence do not coincide. A solution of between 3% and 6% sodium hypochlorite (NaOCI) is commonly used in the hope it can penetrate, circulate and clean into the secondary anatomy if utilized for an adequate period of time. Given enough time it can also digest vital and necrotic tissue fragments that may be harbored in the dentinal tubules or secondary anatomy. However, this irrigation process is very slow and is generally accepted to take at least 30 minutes of direct contact to be efficacious in this complicated anatomy. For many dentists and patients, this process is too time consuming to be clinically effective.

During endodontic treatment procedures, instruments are utilized to shape a canal in preparation for three-dimensional obturation. The by-product of canal instrumentation is the production of dentinal mud. Dentinal mud, in combination with pulpal tissue and bacteria, when present, form what is termed a “smear layer”. This smear layer commonly blocks the dental tubules and secondary anatomy. Blocked lateral anatomy restricts the potential for NaOCI to circulate and clean into the root canal system. The dental profession has long advocated soaking the root canal space with sodium hypochlorite (NaOCI) to encourage disinfection. However, when the dentinal tubules or secondary anatomy are blocked from the incomplete removal of the smear layer, sodium hypochlorite has no opportunity to be in direct contact and hence has little to no effect on those areas. In clinical practice, the results of this disinfection process are unpredictable and time dependent. Endodontic failures are common due to remaining bacteria and/or substrates residual to deficiencies in primary treatment.

Many methods have been advanced to hasten the action of the chemicals used to clean out the contents within the root canal space. These methods include ultrasonic and sonic hydrodynamic agitation, heating, using weak electrical currents, or negative pressure vacuum techniques. Importantly, lasers have also been used in an attempt to improve disinfection. The protocols for laser use have been random and haphazard, and the results unpredictable and non-reproducible.

Laser-target interaction includes reflection, scattering, transmission, absorption and photoacoustic effects. Clinical effects occur through targeting specific tissues and/or micro-organisms utilizing laser energy. When power density is sufficient to achieve the ablation threshold, vaporization of tissue results with minimal collateral thermal damage. Laboratory studies have demonstrated in WO 2004/103471 that achieving high bacterial kill, when using the optimum dye concentration, is energy dependent. The kill level is linearly related to the absorbed energy from a laser energy power source for a defined period of time. Studies have shown that during the laser irradiation of dentin, thermal damage can be minimized by using a highly absorbed laser wavelength and laser pulses shorter than the thermal relaxation time.

Clinical utilization of laser radiation for dental procedures is highly dependent on the form in which the radiation is applied, with respect to the energy level, pulse duration, resting period between pulses, repetition rate, total time and total energy delivered to the target and surrounding tissues. Clinical application of therapeutic radiation dosing must be done in an exact and precise manner relative to all of the variables previously mentioned. Overdosing the radiation delivered can result in temporary or permanent damage to the root and/or surrounding tissues. On the other hand, underdosing results in a lowered or non-existent accomplishment of the therapeutic objectives.

By using lasers, the optical energy can be delivered to the desired area in a precise location and at predeterminable energy levels. The extent to which target is heated, and ultimately destroyed, depends on the extent to which it absorbs the optical energy. It is generally preferred that laser light be transmissive in tissues which should not be affected, and absorbed by the tissues which are to be affected. Non-carious dentin, such as the root dentin is highly mineralized, therefore not likely to be significantly affected by our proposed wavelength range. Therefore, both residual pulpal and pathogenic cells which are largely comprised of water, exist within the confines of dentin and can be precisely targeted and destroyed. Fortuitously, the surrounding highly mineralized dentin, with less water, acts as a natural barrier for the containment of the laser energy. There exists a local peak with respect to water absorption at specific wavelengths in the near-infrared range. In that area of about 980 nm, the energy is the most well absorbed by water. The absorption of water at 980 nm is markedly higher (0.68 cm-1) than at 810 nm (0.12 cm-1) or 1064 nm (0.26 cm-1).⁸

It has also been found that bacteria are “scattered” during high laser repetition rates in excess of 30 pulses per second. Efficient removal of the bacteria can be achieved within a range of 10-25 pulses/sec. Rates below 15 pulses/sec eliminate scattering, but unduly prolong the sterilization process.

It is established that pulsed Nd:YAG (1,064 nm), diode (810 nm) lasers, as well as lasers operating at other wavelengths, will kill pathogenic bacteria, but a quantitative method for determining clinical dosimetry does not exist. A systematic, reproducible method of delivering laser energy to the root canal system in a method controlled in the total amount of energy, its timing, and its distribution throughout the root canal system has not been previously established. Additionally, calculations factoring in tooth type and size need to be made and the corresponding clinical energy amounts/protocols modified to avoid the creation of excessive heat and hot spots within the tooth. For example, lower anterior teeth or the mesial buccal roots of maxillary molars are extremely thin and build up heat rapidly.

The method in which laser energy must be utilized in endodontic treatment is vastly different from the application of laser energy utilized to target other tissues in other procedures. On average, only the coronal ⅓ of the primary root canals can be directly observed using a surgical operating microscope. However, root canal secondary anatomy is extremely small in size, completely random in its location, and is not visible to the clinician at any point in the procedure, even with the aid of a surgical operating microscope. By way of comparison, the diameter of typical accessory anatomy will likely be less in diameter than the period at the end of this sentence. Additionally, the location and contents of the root canal system such as the bacterial pathogen mix and remaining tissue fragments remain unknown to the clinician as well. Therefore, the results of laser treatment in a root canal setting must be inferred, rather than directly observed as in other procedures. Because there is no visual feedback during the procedure, there is no opportunity to modify or correct the location of lasing or its dosing during the procedure itself.

With the advance of the present invention in the ability to deliver larger and better directed laser beams for the described treatments, there remains the possibility that additional shielding of the laser emissions over that provided by the cladding be added. Disclosed below is the further inclusion of a radial shield to be installed over the sheath proximate the limit of the insertion of the optical guide. The shield may be in the shape of a circular disc, centrally disposed over the guide such that when the guide is inserted in the tooth canal, the disc effectively covers the canal such that the bulk of laser emissions are reflected and diffused back toward the tooth and away from the operator.

### Advance of the Present Invention Over Prior Art

While some individual features of components and methodology of this invention have previously been used, it is the refinement of the apparatus, components, processes and protocols, taken in aggregate that defines the scope of this invention. Prior to flight, man, sky, wood, cloth and metal all existed, but until an inventor thought to put them together in aggregate as part of a broader vision, the airplane did not exist.

Current techniques involve either an end-firing or side-firing laser that is inserted into the canal and randomly moved about with the hope that sufficient energy would be delivered in one or more parts of the canal to effect a positive result. The methods in existence today cannot assure removal of all tissue remnants and complete disinfection of the entire canal system.

This invention, in any of the disclosed embodiments, is intended to successfully work with either high-powered lasers (>10 Watts) or low-powered (<10 Watts) primarily diode lasers. Laser emissions may be either continuous or pulsed in either scenario. There are significant differences in the energy calculations for each type of device and its mode of operation.

The correct amount of energy applied and its distribution is essential to the success of this invention and technique. Too little, misplaced, or maldistributed levels of energy result in pathogenic tissue or cells that are not killed, injured, ablated or vaporized, compromising disinfection. The application of too much energy will result in overheating the tooth and/or surrounding tissues, subsequent tissue damage, or possible root fracture. The present technique differs considerably from all other patents in that the described technique is very precise in the following variables: 1) total amount of energy dispensed within the canal system; 2) precise location where energy is dispensed; 3) the pattern of energy distribution; 4) the time over which the energy is dispensed; and 5) items 1 through 4 above relate to experimental values of energy shown to assure both efficacious ablation/vaporization and disinfection/sterilization without direct visualization. Currently described techniques do not collectively recognize the previously mentioned five items. Instead, when held against rigorous scientific standards, prior art involves the incidents of random insertion of the fiber optic tip to a random depth with a random level of energy for a random amount of time producing a random result. The results cannot be relied on as they are anecdotal, inconsistent, non-measurable and nonreproducible.

Uniquely, the wavelengths selected for this technique are specifically chosen to be well-absorbed by water which is the universal component of tissue and pathogens alike. As such, there is no need to utilize a dye to target or mark any given pathological tissue or cells for destruction, though a dye could be used with this technique. If a dye is used to facilitate photoabsorption, power settings and treatment times would need to be adjusted downward. Importantly, the desired wavelengths selected completely avoid the problems associated with the staining agent as enumerated later. Prior systems have not recognized the advantages of the selected band of wavelengths.

This technique allows for the predictable ablation/vaporization of the tissue fragments and micro-organisms left within the primary and complicated secondary anatomy. Residual tissue, bacteria, and related irritants serve as substrates for future reinfection and failure.

Patent Application WO 00/62701 describes, exclusively for caries removal, the basis for photo activity disinfection (PAD). PAD utilizes an appropriate photosensitizing agent to stain, mark, and tag bacteria. Upon irradiation with a laser, the interaction between the laser and the dye leads to singlet oxygen release and results in the death of the bacteria. This technique makes no mention of the need for removal of the substrates of the bacteria to prevent future infection. The technique described in this application, by contradistinction, requires no dye and directly targets the essential ingredient of all living cells, namely water through proper selection of alternative and appropriate wavelengths. is publication describes a tip which is shaped to spread light around an arc of up to 360 degrees at a specific geometric plane. Importantly, this publication describes a method for caries removal and not endodontic disinfection/sterilization. The present invention will fire radially along the length of the fiber, and in multiple geometric planes. Alternative embodiments will fire in 360 degree bands which can then be moved to successive levels.

In further contradistinction the invention described in WO 00/62701, no use of an isotropic tip is contemplated that is generally spherical and in the small micro-sizes required to fit into a root canal preparation. However, in larger canal applications, such a use is possible but not necessary.

Publication WO 00/62701 also briefly describes another way to form an isotropic light-emitting tip by removing the internally reflective outer layer of the optical fiber over a short distance from the distal end, or by restricting the outer layer so that it is not applied to the distal end.

In contradistinction to U.S. Pat. No. 5,092,773 which relates to the use of laser radiation for treating mineralized body tissues, the presently described invention is specifically designed to treat bacteria and soft tissues contained within the confines of the root canal space, the periodontal ligament and tissues immediately adjacent to the exterior root surface.

### OBJECTS OF THE PRESENT INVENTION

Within the interior of each tooth exists a system of channels and tunnels housing the dental pulp. This system consists of larger primary canals (primary anatomy) and a system of smaller interconnected branches, fins, loops, webs, tributaries, cul-de-sacs, anastomoses and other smaller irregularities called the secondary anatomy or accessory anatomy (See FIG. 11). These form the primary anatomy and the secondary anatomy, in combination, are referred to as the root canal system. This system, similar to a fingerprint, is unique to each individual and unique to each individual tooth. No two root canal systems are alike and the exact morphology is never known to the clinician in advance of treatment. Accessory anatomy can occur anywhere along the length of the primary canal and in any form or combinations of forms. There are several situations in which the present invention has particular application including:
1) Disinfecting/sterilizing root canals.
2) Ablating/vaporizing biofilms, necrotic debris or vital tissue within the root canal system.
3) Controlling the amount of energy applied to the root canal system and the precise control of the location and distribution of said energy application.
4) Disinfecting the periradicular external root surfaces of a tooth both internally from the prepared canal, or externally by surgical procedures.
5) Removal of root canal filling materials or obstructions including broken instruments.
6) Removal of carriers in previously treated carrier-based obturations.
7) Anesthesia of unanesthetized pulpal tissue by direct application of a controlled amount of laser energy to the pulp or pulp fragment.
8) Repair of cracks and root fractures
9) Treatment of root resorption defects, both internal and external.

In direct contrast, clinicians performing procedures other than endodontic procedures have direct visual confirmation of the results of the application of the laser energy. Specifically, clinicians can visualize the procedures and energy application results directly in real time. They can also and monitor and modify the application of the correct amount of energy and see when the application of laser energy has been sufficient to accomplish the desired task—again in real time. Endodontic disinfection/sterilization procedures are different in that they are done “blind” and the clinician can never see the results of the laser irradiation and hence has no visual confirmation to determine if the complete root canal system has been three-dimensionally cleaned and all tissue fragments removed—even after treatment has been completed. Again, treatment results in endodontic applications must be indirectly inferred while treatment in other tissue applications may be directly observed. In order to infer a successful result, the clinician must be able to precisely control a number of factors including the power of the energy pulse, time of the energy pulse, time of rest between pulses, the total levels of energy delivered to the root canal system, the placement and distribution of that energy within that system and the total time of exposure. These factors and values must then be compared with experimental and scientific norms required to accomplish disinfection of the root canal system. In many respects, the process is similar to sterilization procedures with an autoclave. One does not get to visually confirm that the bacteria, spores and viruses have been killed, one infers that they are destroyed based on following rigid protocols and periodic verification tests.

Like an autoclave, insufficient levels of energy delivered throughout the canal system will result in incomplete bacterial kills, or inadvertent remaining tissue irritants which will result in continued bacterial infection or promote re-infection at a later date. Once again, the long-term success of endodontic treatment often fails due to remaining bacteria or their substrates in the root canal system.

Even to “guess on the safe side” by leaving the activated laser tip in the prepared canal for a longer period of time or needlessly increasing the power may result in an unacceptable and uncontrolled level of heat generation with subsequent tooth or surrounding tissue damage. As the state-of-the-art exists at the moment, the clinician must either “under guess” or “over guess” the endodontic energy requirements. The option to correctly apply and distribute an effective, safe, and calculated amount of energy into the endodontic space simply does not exist in today's environment. Without direct vision, an evidence-based method and apparatus utilizing scientific validation is necessary in the application of laser energy in endodontics.

Historically, the lasers used to attempt some form of endodontic treatment of the root canal system have used wavelengths in the range of 600 to 810 nanometers. These wavelengths are poorly absorbed by water. The current invention has been designed to do the exact reverse of that concept. The present invention is designed to specifically target high water content of cells and leave the surrounding highly mineralized tissues healthy. Previously, for energy absorption to occur in sufficient quantities to assure some form of satisfactory bacterial kill, the targeted cells in the prior art had to be first impregnated with a dye. The dye served to attract the radiated energy as well as act as a heat sink for that energy to target specific micro-organisms. The interaction between the laser and the dye leads to singlet oxygen release and results in the death of the bacteria which is the basis of photoactivating disinfection (PAD) therapy.

Previous inventions have modified a traditional end-firing laser fiber to fire laterally. No mention was given to the dilution effect the side-firing embodiments had relative to the lost energy to the end of the firing tip. A laser's energy is most effective in its highly coherent, end-emitting tip. Side-firing or radial-firing lasers will dilute energy and the effectiveness of end-firing lasers. As such, side-firing emission creates different zones of variable energy, both laterally and at the most distal end-firing tip. The method in which the side-firing action is accomplished will directly influence the amount of energy available both along the lateral surfaces and to the most distal extent of the fiber.

The interaction of laser energy with the target tissue is mainly determined by the specific wavelength of the laser and the optical properties of the target tissues. Total energy delivered, power density, energy density, pulse repetition rate, pulse duration, time of rest between pulses, and the mode of energy transference to the tissue can be easily controlled by the clinician. Combinations of these factors serves to control the optimal response for the clinical application. When the laser beam hits the target tissue, reflection, absorption, transmission and scattering can occur. Three main mechanisms of interaction between the laser and the biological tissues exist: photothermic, photoacoustic and photochemical. The effect of lasers is based on transformation of light energy into thermal energy which, in turn, heats the target tissue to produce the desired effect.

There exist several differences between high-powered, free-running pulsed (FRP) lasers and low-power diode lasers that bear directly on the mechanisms of action. The corresponding clinical considerations for this invention warrant acknowledgement and discussion.

Diode lasers are very different from FRP lasers. FRP lasers generate very high peak powers in very short time periods which allow for heat dissipation. Diode lasers do not. The generation of heat with a diode laser during treatment is a significant clinical consideration. FRP lasers may be used to remove tissue essentially without constraints of time or heat buildup and subsequent tissue damage while the diode laser cannot.

In contradistinction to a FRP Nd:YAG laser, a diode laser, in either continuous wave or pulsed/gated configuration, does not have the high peak power or microsecond pulse capability of the FRP Nd:YAG laser. A diode laser has far longer pulse durations with far less peak power that will not reach the ablation threshold in soft tissues.^{[1] [2]} Instead the output power is converted primarily to radiant heat energy requiring a different dosimetric approach than for the FRP Nd:YAG lasers.

Because of the differences previously described, the diode lasers generally work by contact vaporization while the Nd:YAG lasers work by ablation. The diode laser will cause a larger amount of energy to be converted to local heat at the fiber tip. Because of the rapid heat generation and buildup produced by its method of operation, the diode lasers allow for much smaller margins of error. It is essential when using diode lasers in the root canal system to develop a method of precise timing, calibration and distribution of the energy delivered.

Diode lasers can, upon activation and contact with tissue, carbonize at the tip, dramatically changing its working properties. Because of the damage to the fiber optic tip due to carbonization of the intracanal contents, any defined beam area is eliminated and the energy is converted to local radiant heat with the fiber tip rapidly becoming “red hot”.^{[3] [4]} This heat energy is then transferred to the contents within the canal via thermal conduction and works via contact vaporization versus the true ablation of the FRP Nd:YAG laser.

The thermal conduction of the diode laser is a fundamentally different mechanism of energy transfer than is seen with a FRP Nd:YAG laser. Additionally, the high peak power pulses of the FRP laser help ablate and remove debris caught on the Nd:YAG fiber tip, which would otherwise block the forward laser emission and produce a buildup of heat in the fiber^{[5],} Clinicians should be aware when using a diode laser that changing from a non-contact mode to a contact mode of application greatly influences the resulting effects because of the carbonization of the tip and the subsequent rapid buildup of heat at the fiber tip.

Myers^{[6]} suggested specific dosimetry computations for the application of laser energy applied to periodontal pockets with an Nd:YAG laser. These computations related to work performed outside the confines of the root and did not involve the root canal system. His work generated a dosimetry table based on the probing depth of the pocket to be treated. This work led to the first FDA market approval for “laser sulcular debridement”.

Subsequently, Gregg and McCarthy^{[7]} created a computation defining the quantity of laser energy delivered to the treatment site of periodontal pockets. These calculations then allowed for comparison of different laser systems examined in similar studies.

To compensate for the heat produced by diode lasers, the traditional dosimetry equations used for the FRP Nd:YAG lasers must be altered and treatment times developed that assure a comprehensive effect on the target cells and tissues while avoiding unwanted thermal tissue damage to untargeted tissues. Clinical modifications necessary to ensure safety and unwanted tissue damage will include measurement of the energy delivered over time, lowering the total energy delivered into targeted area, and precise control of the site of the energy phasing. These specific alterations are necessary because the diode laser carbonized tip does not have a “beam area” for the incandescent hot tip. Without a defined beam area, there can be no accurate energy calculations.

While many operators will dry the canal at the end of the procedure prior to lasing with ethyl alcohol, it is strongly advised that this not be done prior to the use of the laser as outlined in this protocol as the alcohol will ignite and depending on the amount of alcohol present will either smoke, flash or burn. Its use is unnecessary with this technique in that the heat from the laser will dry the canal on its own.

It should be noted that the invention and its embodiments relate, in large part, to the ability to determine the amount of energy dispensed, its placement, timing and distribution and hence can be used with FRP Nd:YAG lasers as well as other lasers of most wavelengths. It and its embodiments may also be used with energy absorbing, targeting dyes as well (PAD). The difference is that the power settings and exposure times will need to be recalculated on an individual basis—most likely downward in the case of the FRP Nd:YAG and targeting dyes. Experimentally, the power settings for a diode laser need to be considerably less than that of a corresponding FRP Nd:YAG laser.

### SUMMARY OF THE INVENTION

This invention includes unique concepts, protocols, apparatuses, and clinical applications as well as new and unique methods for preparing the root canal system for use of the apparatus. The embodiments of this invention fall into two broad supracategories—“energy phasing” and “energy distribution”. The first supracategory classification is determined by whether laser energy is delivered in “phases” to portions of the canal or the energy is delivered to the entire canal at once in a single treatment “phase”. For purposes of clarity, these two embodiments shall be referred to as “energy phasing” embodiments, i.e. the total energy is delivered clinically in stages, or all at once.

The second supracategory relates to the method and location of energy distribution accomplished by the modification of the actual working portion of the fiber itself. These will be referred to as “energy distribution” embodiments. Various embodiments can be then developed by combinations of elements from each of the supracategories. For example, if there are two energy phasing embodiments, A and B, and there are 8 energy distribution embodiments¹¹-⁶¹, then combinations thereof produce A1-A8 and B1-B8 embodiments.

The apparatus is a disposable laser fiber tip capable of side-firing or radial-firing in such a way that the amount of energy is controlled along a part of or the entire length of the radial-firing part of the fiber as well as the tip. For the purposes of this application, side-firing and radial-firing shall be used interchangeably and shall mean any emission of laser irradiation at an angle of between 1 degree and 360 degrees from the long axis of the fiber. The fiber optic tip would radiate energy around at varying angles producing essentially a distribution of energy arranged in essentially a cone formation along the long axis of the fiber. This would be accomplished by creating slits or other openings in the cladding and exterior reflective coating of the transmitting fiber. The slits/openings would allow the emission of a prescribed, calculated amount of laser energy at precise locations.

Previous techniques require the use of a dye to pre-stain the targeted tissue and pathogens to preferentially absorb laser irradiation in the approximate 600-830 nanometer range which is poorly absorbed by water. These wavelengths were ineffective in targeting the water of living cells and consequently dye was necessary in the PAD method to get the cell membrane or cell body to absorb enough energy to produce the desired effect. In contradistinction, the present invention directly targets water, a ubiquitous component of all living systems including bacteria, yeasts and viruses. The inventive laser technique of this embodiment uses the frequency of the wavelength emissions between about 930 to about 1065 nanometers with an optimum of 980 nm. This range of wavelengths is designed to specifically target the water content of tissue cells and pathogens as well as any residual organic debris in water within the root canal system after its preparation while being poorly absorbed by the surrounding dentin. The selection of the optimum wavelength produces significant effects (described by some in the dental laser application as photoacoustic effect) as well, particularly in the targeted aqueous environments. This is due to the rapid energy absorption by the water and the subsequent creation of gas bubbles, liberation of heat and subsequent propulsion of waves of heat and gas that impact along the canal walls and ramifications resulting in an enhanced bacterial kill and cleaning of the canal walls and ramifications. No dyes or other additives are utilized to enhance the effectiveness of the laser kill of bacteria, etc.

This technique avoids the need to use a dye and therefore avoids the problems associated with the use of dyes. Such problems include confirming the dye can even reach the desired target due to dentinal mud, blockages, or complex anatomical challenges. Additional problems include excess dye deposition which impedes the bacterial kill rate, time to apply and wait for uptake, storage, inventory, removal of all dye traces prior to esthetic restorations, staining teeth, uncertainty of even application, allergic reactions and the general mess and care of handling dyes.

Endodontic biofilms, a target in this protocol, are protected by a sticky exopolysaccharide matrix that protects the microbes within from antimicrobial agents (antibiotics), the immune system, or endodontic reagents utilized in treatment. A large portion of the canal contents needing to be removed by endodontic treatment are proteins. Proteins change their properties with the application of heat. For example raw egg white, versus cooked egg white, would much more difficult to remove from the canal. The goal is to accomplish a phase change in protein structure to enhance removal after the kill. The application of the laser energy to effect the denaturization of proteins such as tissue fragments trapped within the ramifications of the canal system results in the deprivation of acceptable substrate for the continued viability of bacteria. The bonds of the denatured protein substrate broken and their energy released robs the bacteria of the energy needed to sustain themselves. It is the equivalent of bacteria trying to live on a diet of ash. This has two effects. One, there is no energy in their food. Two, ash creates an alkaline environment which is generally hostile to bacteria. Therefore, even without the complete removal of all tissue fragments within the canal, one can significantly enhance the therapeutic outcome by the denaturization of proteins within the canal and its ramifications, even if they are not removed completely. The dramatic effect of the rapid absorption of the generated heat contained in the light of the appropriate wavelength by the water causes the release of steam and gases from the evaporation and transformation of the bacteria and tissue produces a wave-like effect as these advance through the interstices of the canals. The impact resembles the physical impact of such as a storm-surge of a typhoon or hurricane which promotes the cleaning of the canal walls. This effect is a startling discovery in the use of the low power, limited wavelength laser application causing the disclosed inventive system to provide superior treatment with a lower cost, low power diode system.

The clinical assignment and goal of this protocol involves the controlled released of energy versus the random application of laser energy within the root canal system. Energy release is controlled both in the total amount of energy delivered to the canal as well as the time, location and distribution it is delivered in the canal. These settings are determined from experimental research showing that such times and energy levels are sufficient to assure the ablation/vaporization of the biofilms, tissue cells/substrate and bacteria harbored inside the root canal space and root structures.

### BRIEF DESCRIPTION OF THE DRAWINGS

FIG. 1 is a side view of an endodontic laser head and tip for disinfecting and sterilizing and/or disinfecting the internal root canal anatomy of a tooth.

FIG. 2 is a cross sectional view of an alternative tip of the laser of FIG. 1.

FIG. 3 is a cross sectional view of an another alternative tip of the laser of FIG. 1.

FIG. 4 is a cross sectional view of an another alternative tip of the laser of FIG. 1.

FIG. 5 is a cross sectional view of an another alternative tip of the laser of FIG. 1.

FIG. 6 is a cross sectional view of an another alternative tip of the laser of FIG. 1.

FIG. 7 is a cross sectional view of an another alternative tip of the laser of FIG. 1.

FIG. 8 is a cross sectional view of an another alternative tip of the laser of FIG. 1.

FIG. 9 is a partial side view of a tip with a spiral emission slot.

FIG. 10A is a cross sectional view of a tooth showing insertion of the of the laser of FIG. 1.

FIG. 10B is a cross sectional view of a tooth showing the insertion of the laser of FIG. 1 in a broken tooth.

FIG. 11 is a cross sectional view of a tooth showing the anatomy of the tooth.

FIG. 12 is a block diagram of the endodontal laser of the invention showing the operating components.

FIG. 13 is a side view of an alternative embodiment of laser head and tip incorporating a radiating window with an axial orientation in relation to the optical guide.

FIG. 14 is a front view of an endodontal laser tip having a shield disposed proximate the tip.

FIG. 15 is a cross sectional view of a n alternative embodiment of a laser tip according to the invention wherein the tip has a slidable shield axially thereon.

FIG. 15A is a cross sectional view of the laser tip of FIG. 15.

FIG. 16 is a sectional view of a clad fiber according to the present invention.

FIG. 17 is a further alternative view of the fiber of FIG. 15.

FIG. 18 is a sectional view of the use of a reflective stop for transmitted light energy.

FIG. 19 is a pictoral view of the stop of FIG. 18.

FIG. 20 is a pictoral view of an alternative embodiment of the stop of FIG. 18.

### DETAILED DESCRIPTION OF THE PREFERRED EMBODIMENTS

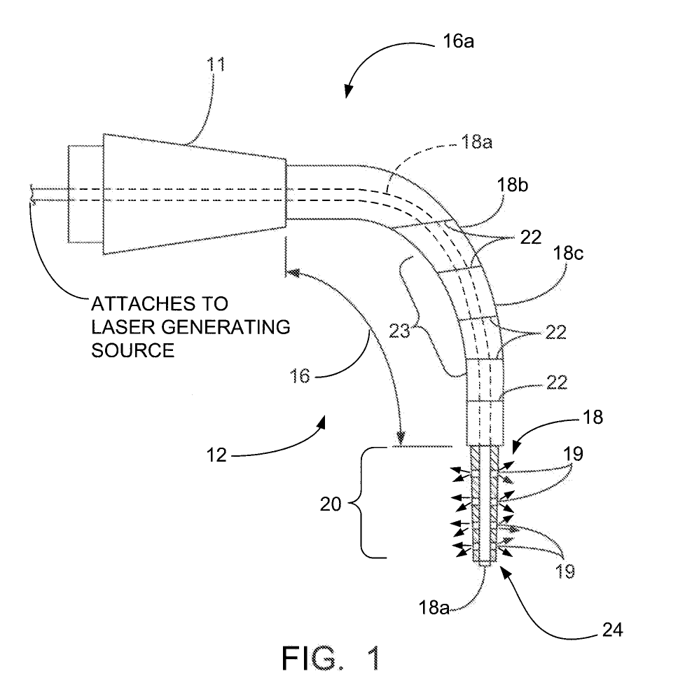

The apparatus is a flexible disposable laser fiber tip 12 capable of three-dimensional side-firing or radial-firing along its working length. See FIG. 1) The working length is defined to mean the portion of the fiber that emits laser energy for the purpose of doing work. It may include an end-firing tip, radial or side-firing emissions, or a combination, thereof. The actual working length is determined by the modifications to the protective and reflective coverings surrounding the transmission fiber. It is anticipated that the diameter of the working fiber, including coverings, shall have an external diameter of about 200 to about 800 microns but may be smaller as manufacturing techniques allow. Further, the working fiber may be parallel or, alternatively, may have either a fixed or progressively percentage change taper over its working length. Clinical laser apparatuses will embody different working lengths and sleeve configurations to accommodate the particular requirements of clinical needs. The control of the energy release along the active tip is accomplished in different ways to achieve preferred levels of energy release as subsequently described.

As mentioned above, the present invention relates to a laser apparatus for effective endodontic procedures not previously available. The present inventive apparatus is in part directed to the special laser beam emission tips which provide measured irradiation of selected portions of the primary and secondary channels of the tooth. Referring now to FIG. 1, one embodiment of the tip apparatus is illustrated. Tip 12 is connected to a laser source (shown in FIG. 10) via head 11, later illustrated and described. The source is a conventional laser generator and guide tube, however operating at the unconventional wavelengths described. In preferred embodiments, the laser source is a diode laser. The source is programmed to provide the particular wavelength and irradiation patterns embodied in the described apparatus and methodologies.

As illustrated in FIG. 1, tip 12 includes a fiber optic tip and sheath 16 making up the guide 18, including the fiber optic bundle 18a, the cladding 18b, and an optional protective layer 18c, for carrying the laser beam to the delivery region 20 of the tip 12. The upper flexible sheath portion 16 optionally includes a plurality of calibration or depth markings 22 whereby the user may select the depth to which the energy release is delivered to a region disposed in a channel. (see FIGS. 10A and 10B) Sheath 16 additionally includes color coded firing (timing) bands 23 which may indicate relative amounts of energy to be delivered to associated portions of a canal. As further illustrated in FIG. 1, fiber optic guide 18a extends into the delivery region 20 whereby emission of the laser beam may be selectively directed to predetermined areas of the primary canals. (See also FIGS. 10A and 10B)

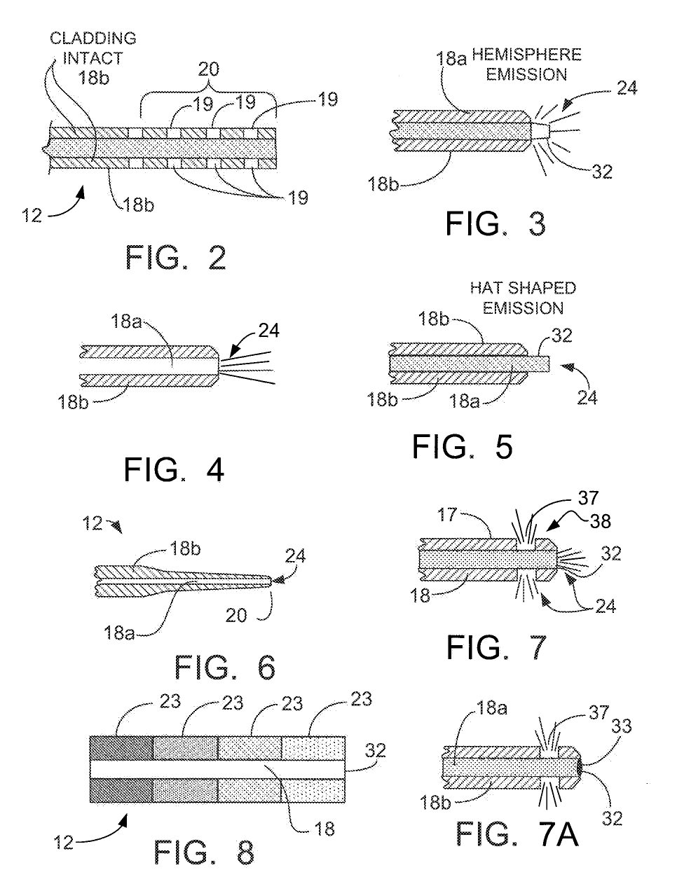

Further, in the described and illustrated embodiments, FIG. 2 illustrates a tip 12 having a working length making up emission area 20 wherein the portion of the guide 18b extending from sheath 16 incorporates a slotted reflective coating/cladding 18b′ allowing a limited release of energy through emission windows 19. Slotted reflective coating/cladding 18b′ is in the form of a circumferential opening in the reflective coating/cladding which may exhibit a 360° opening or a fraction thereof. Workable widths of the openings are from about 0.2 mm to about 5 mm and in numbers of bands of from about 1 to about 8.

FIG. 3 illustrates a tip wherein the cladding sheath 18b extends fully to the delivery region 32 at the end of the tip 12, wherein the sheath 18b terminates adjacent the end of the guide 24 however, exposed sufficiently to produce an emission pattern resembling a hemisphere. To accomplish such a pattern, the exposed guide may be on the order of about 0.2 mm to about 3 mm including a tapered or rounded aspect at the exposed portion. An emission pattern of this style is particularly useful for procedures including treatment of the most apical primary and secondary anatomy.

The embodiment of tip 12 illustrated in FIG. 4 contains a cladding 18b of sheath 16 extending integrally to the distal end (delivery region 24) such that the emission from guide 18a is axially out of the end of the guide. An alternative embodiment (FIG. 7) of this style of tip 12 may include a single circumferential window 37 adjacent the distal end 38, the window 37 having a width of from about 0.2 mm to about 3 mm and positioned from about 0.1 mm to about 3 mm from the distal end of the tip 38. An emission pattern from this style of tip is particularly useful for procedures including treatment of the most apical primary and secondary anatomy.

The embodiment of tip 32 illustrated in FIG. 5 provides an end-firing tip, wherein the energy irradiation pattern is effectively “hat-shaped”. the cladding or sheath 16 surrounding the light guide 18a to provide a significant end-fired working beam which provides side-firing at the tip 32 as well as axial firing.

The embodiment of tip 12 in FIG. 6 incorporates a layered cladding 18b beginning at a predetermined point approaching the delivery region 20, where the thickness of the cladding gradually decreases to zero such that the radiated energy gradually increases through the delivery region to a maximum level at the distal end of the tip 24.

In the embodiment of laser tip 12 illustrated in FIG. 7A, cladding 18b extends to the tip 32 of the guide and includes a cap 33 over the end of the guide 18a to block axial release of energy. Alternatively, the energy release is through windows or slots 37, similar to those in FIG. 7.

In the embodiment of an alternative to the tip 12 of FIG. 1, FIG. 8 illustrates bands of a color coded cladding 23 disposed over guide 18a to provide depth indication to the user of the tip 12 as it is lowered into a canal.

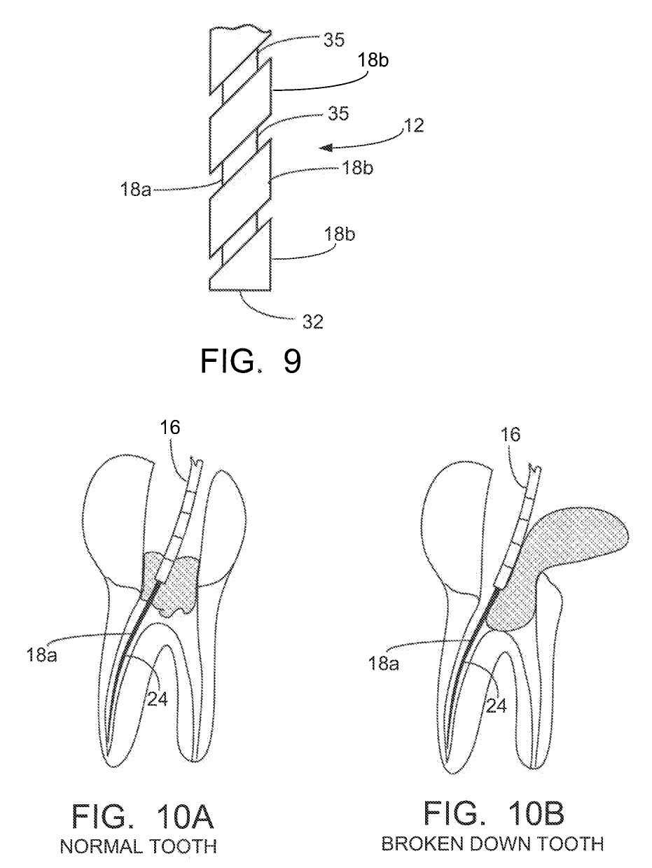

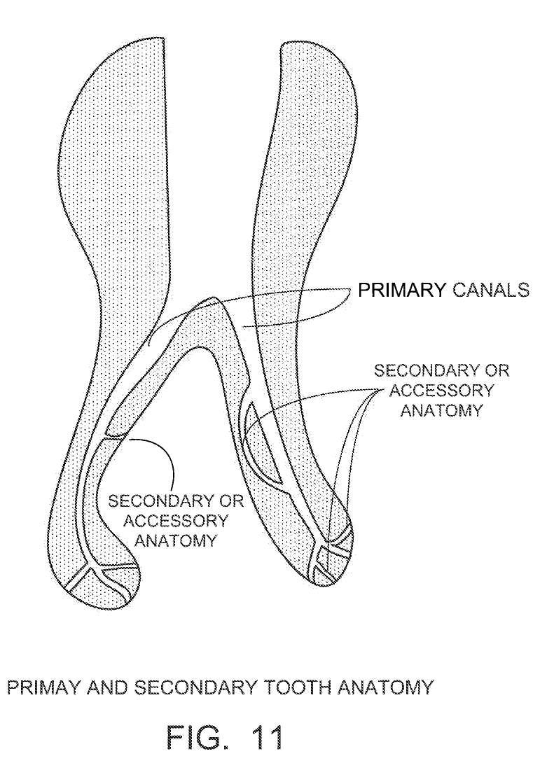

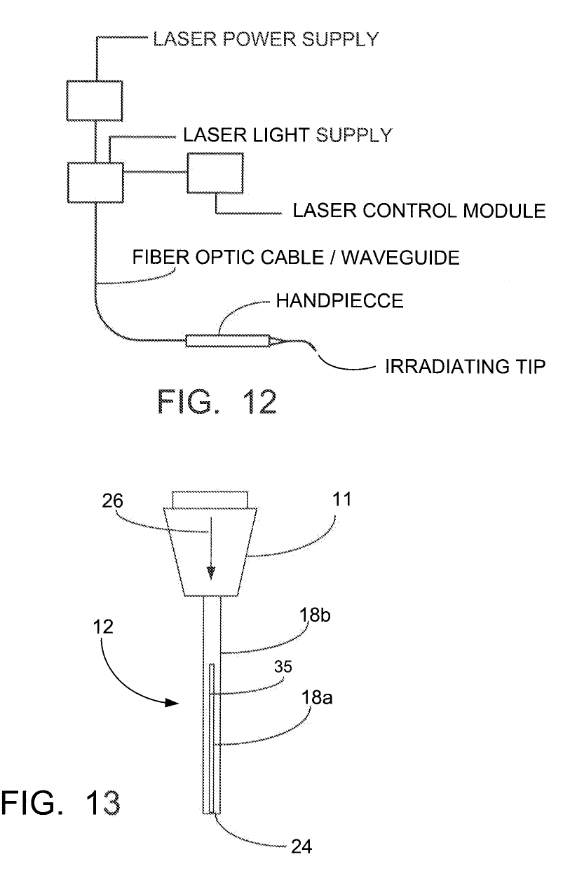

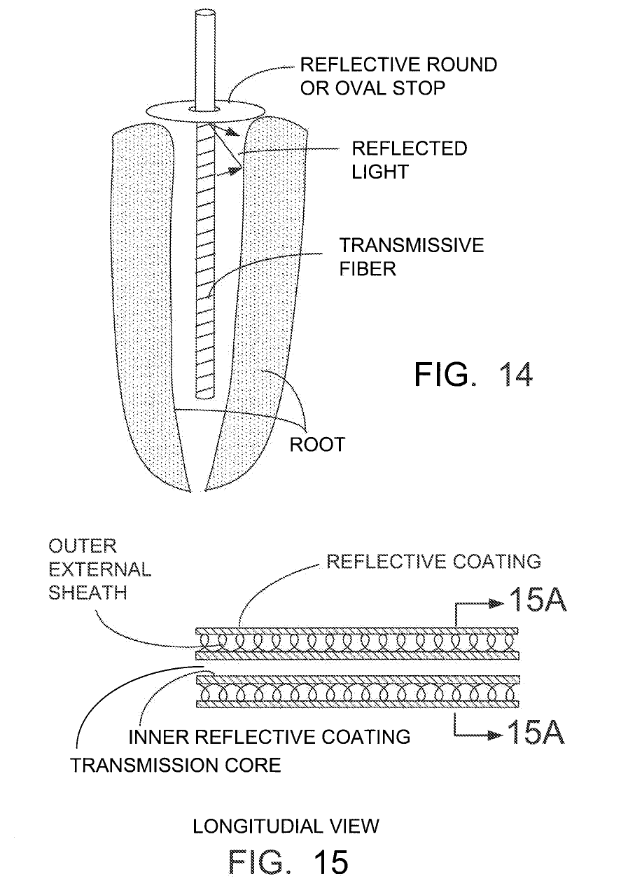

FIG. 9 illustrates an alternative tip 12 wherein the emission window 35 comprises a helical spiral over the emission region 20 to the tip 32.

FIG. 13 illustrates another alternative embodiment of tip 12, wherein an axial window or slit 35 in the cladding 18b extends from a predetermined distance from a selected point below the head 11 to the distal end 24 of the tip. This embodiment may incorporate a single or multiple radiation windows, including such as two windows spaced 180 degrees around the sheath 12, or windows at other uniform (120°, 90° locations) or grouped regions. such as two or three windows within a 45° span of the cladding 18b on sheath 16. An index marker 26 may be disposed on head 11 to indicate the relative position of the radiation window 19

The method of how energy is measured, controlled and distributed in this application is very important. The energy release is regulated in such a way that the amount of energy released is controlled along a specified part of, or along the entire working length of, the radial-firing part of the fiber as well as at the tip. The configuration of such controls is a function of the intended clinical outcome. It is projected that about 200 Joules total energy administered at a wattage of between 0.5 to 2.0 watts in short increments, their exact time calculated dependent on the wattage, tooth type, length and thickness each followed by an approximate 15 second resting period should be sufficient to assure disinfection of the root canal system without overheating the tooth or surrounding structures. Release of energy may be in pulses of specific duration and/or energy level. Likewise, the energy may be delivered in patterns of numbers of such pulses a selected pulse levels and duration, as may be particularly effective for certain treatments.

The energy formula: [(units of energy released over time)×(the total time of release)=the total amount of energy released into the root canal system] is both measurable and reproducible and is a function of the time spent in the root canal system with the irradiation turned on less the small allowance for waste energy. It is the specific control and quantification of laser irradiation emissions over time at a specific location that allows assurance of target tissue and cell destruction. This laser irradiation within a prepared canal occurs without concurrent direct vision of the results and must occur without excessive heat buildup that would damage the non-targeted and surrounding tissues including nerves, blood vessels, dentin, periodontal ligament, bone and soft tissue. The present invention, by targeting the water contained within the canal, whether absorbed or contained within unwanted bacteria, diseased tissue or debris, enables the generated heat (from a low power source) to be efficiently focused and absorbed by the water, as opposed to the adjacent tooth structure thereby providing a safety factor to tooth destruction. Likewise, the ability to focus the heat generation in the contained water promotes the “wave effect” of the rush of the heat, gas, bubbles and like products of the more rapid heating than provided by other systems.

Previous attempts at laser use do not have protocols for precise control of the total energy delivered, location of energy phasing, distribution, or time of delivery, thus they cannot be both predictably efficacious and safe Importantly, existing protocols do not address the different energy needs by tooth zone. Current protocols are usually done as the random application and movement of a point source for an indeterminate amount of time without strong scientific data supporting the results of these current nonquantifiable approaches.

### “Energy Phasing” Embodiments

In endodontic treatment, it is the specific control of laser irradiation emissions that allows assurance of target tissue and cell destruction without excessive heat buildup that would damage the non-targeted surrounding tissues. The energy phasing control mechanism may be of several embodiments.

In the first embodiment (FIG. 1), a depth gauge 22 is incorporated in a cladding sleeve/sheath around part of the fiber housing that allows for partial irradiation of the root canal in specific treatment zones. The illustrated embodiment illustrates slots 19 in the cladding for the radial, side-firing energy release. At the tip 24, the cladding stops short of the end of the fiber optic guide 18 permitting 360° energy release. When the appropriate amount of energy has been delivered, the tip is manually moved to a new zone indicated by the color-coding on the sleeve/sheath or cladding of the fiber. The zones are typically from about 3 mm to about 7 mm in depth. The markings should be such that a dentist may readily identify the depth of insertion of the tip of the instrument. The cladding, sleeve/sheath and working area of the fiber should be of such a configuration as to prevent the irradiation much beyond 1.5 mm inside of the canal proper, particularly at the apical constriction. This protection may be accomplished by the selection of a sleeve of correct length, including such as a telescoping sleeve, a movable sleeve—with or without windows allowing lateral emission of energy, removable sleeves of different lengths, or rings of additional sleeve/sheathing material that can be added to effectively extend the length of the sleeve. This precaution is to prevent stray radiation from injuring surrounding tissues or the clinician, staff, and patient. This shield can be very important in badly broken down teeth where the working portion of the fiber is no longer completely surrounded by tooth structure.

Another preferred embodiment (FIG. 9) is configured whereby the laser fires 360 degrees horizontally along the entire working length of the fiber via a helical spiral slit 39 in the reflective coating/cladding 18b originating at the top of the working length of the fiber and ending at the apical tip. Such a helical slit shall be between 0.05 mm and 1.5 mm wide and shall make between one and four complete revolutions around the fiber at the tip. The slit width and helical configuration are not designed to impart either flexibility to the fiber nor change their dimensions on flexion in contradistinction to US2004/0038170 and U.S. Pat. No. 7,040,892. The spiral winding or the slit width may not be uniform along its length allowing for its tighter winding or a wider slit at areas where an increased delivery of energy is required and a looser winding or narrower slit where areas of less energy is required. This configuration allows for a “three-dimensional or 3-D lasing” of the inside of the canal. Its energy phasing is controlled both in time and emissions by an electronic device. The device advises the clinician when the appropriate level of energy has been dispensed. In this way the clinical delivery is most efficacious treating one canal at a time in a single step procedure for a prescribed amount of time and without the need for staged movement of the laser tip. Such a tip should be inserted to a depth within one mm of the confirmed working length for the canal to be treated. As mentioned earlier, alcohol, chloroform or flammable liquid of any type should not be present at this point. The energy delivered to the selected canal should be approximately 200 Joules delivered be delivered at a low wattage as previously described with integral resting periods of about 15 seconds each in which no energy is delivered into the canal to allow the root to cool down. Halfway through the treatment interval, an audio alert will sound and the tip should be moved coronally the thickness of one or more color indicator band(s). The width and exact dimensions of such band(s) shall be calculated in accordance with the energy distribution of the radial slit. By moving the tip the appropriate distance, the reciprocal, untreated areas may be effectively irradiated while allowing the recently treated areas to cool down. The process is then repeated until the total 200 Joules has been delivered to the treated canal. To further enhance both the disinfection and cleaning of the canal, one can fill the canal(s) with an aqueous solution and activate the tip again at a low wattage of between about 0.5 to 2 watts for short periods of time followed by resting periods to take advantage of the photoacoustic effects of this device.

The third embodiment (FIG. 3) is a variation of the first embodiment and preferably includes such as electronic time and power controls whereby the clinician moves to a new treatment zone after the appropriate energy for bacteria, etc. kill has been delivered to the first treatment zone. The tip radiating portion is a 360° section at tip end 24 wherein the radiation beam extends about 3 to about 7 mm beyond cladding 18b.

In another embodiment (FIG. 8), the color-coding/gradation concept may also be applied directly to the out fiber cladding itself to achieve the same purpose. Energy distribution control may be accomplished by any of the four embodiments previously listed.

In general, side-firing of a laser fiber may be accomplished by a variety of means (See FIGS. 1 through 6). The two methods deemed most feasible for this application include the calculated circumferential scoring of reflective coating/cladding of the fiber which allows a radial or lateral 360 degree distribution of the laser energy from the scored areas. An alternative embodiment for energy distribution is from a tip wherein the reflective coating/cladding thickness is varied from full occlusion to a zero, or nominal, level at the distal end of the tip. Such may be achieved by etching of the cladding by dipping the fiber and its reflective coating/cladding into a strong acid and the timed withdrawal of the fiber from that acid yielding a gradient of exposure through the reflective coating/cladding (FIG. 6).

In the first energy distribution embodiment related to the fiber scoring, the controlled release of energy is produced in one or more bands along the length of the active fiber tip. The purposes of releasing the laser energy in bands are to first adapt the technique to lasers of low power where there is not enough energy available to produce effective energy release along the whole working length of the fiber tip. Second, releasing laser energy in bands also serves to more finely target the energy release in the zones deemed to be of particular therapeutic interest and to reduce the total amount of heat absorbed by the root and surrounding tissues. Energy bands released from the fiber may be uniform in thickness, not uniform in thickness, or graduated depending on the clinical needs of energy release. The energy emissions from the working tip may also be partially or completely blocked at its most distal terminal extension to reduce or completely eliminate energy emanating from the tip. Such capping may be of value when operating around delicate anatomical structures or to conserve, or redirect energy flow to its more proximal side-firing counterparts.

In the second energy distribution embodiment, the controlled release of energy is accomplished along the entire three-dimensional working length of the fiber and all areas are fired simultaneously. Total energy delivered is calculated and monitored from the laser source with appropriate safeguards for over and under-exposure. The laser tip 12 is designed to deliver sufficient energy to achieve the desired outcome but importantly, the energy must be controlled to prevent destroying delicate apical root canal anatomy which could complicate treatment or retreatment efforts, if necessary.

The third energy distribution embodiment is the calibration markings of the sleeve that houses the laser tip or the calibration markings are placed directly onto the external aspects of the fiber cladding or reflective coating itself (See FIGS. 1 and 4). Such markings may be calibration markings, numbers and/or color-coded bands of clinical significance. Such markings are sized to incorporate a direct energy release relationship to the disinfection/sterilization energy requirements for that zone depth. Such markings may be used in conjunction with time measurements to coordinate the movement of the active tip after a predetermined amount of energy has been dispensed. Endodontic applications will require that this sleeve 18 be bendable/flexible so the laser fiber and sleeve/sheath can be curved to more than a 90 degree angle. Clinical access and usage requirements dictate that it is a requirement that the insertion of the disposable tip into the handpiece be able to be rotated 360 degrees at the junction 26 with the handpiece (FIG. 1). It is most likely that the features of the described first embodiment will be included with the third embodiment (FIG. 2) to create a tip that would fire in zones, such that the zones would overlap slightly upon removal of the tip, ultimately dosing the entire root canal system over the controlled withdrawal of the tip. Variations in scoring methods for energy distribution embodiments are further illustrated in FIGS. 1-9.

It is envisioned multiples of the disclosed series of tips may be used in clinical practice. The first tip is an end-firing tip used to treat the apical region of the canal (FIG. 3). Its configuration and energy release are such be such that it will not iatrogenically damage the delicate apical anatomy and yet produce emissions designed to penetrate the apical portion of the root to exert its effects on pathogenic micro-organisms residing on the outside surface of the root and in the surrounding tissues.

In addition, there can be different styles of side-firing tips (See FIGS. 2, 4, 5 and 6). Another side-firing tip is “end capped” (FIG. 7A) in such a way that no emissions are produced at the tip as would be the case in an “end-firing” embodiment. The construction of this design allows for irradiating the canal without producing emissions directly out the apical end of the root. This embodiment is selected in cases where delicate anatomical structures (neurovascular) approximate the root end. In another embodiment (FIG. 2), the side-firing tip could have an apical end-firing component as well.

While the circumferential openings in cladding 18b, whether as illustrated in FIGS. 2 and 4, provide useful diode laser delivery mechanisms, it is also within the scope of the present invention to utilize longitudinal, or axial slots 35 as is illustrated in FIG. 13. In this embodiment, the slots forming the openings for axial radiation may be as narrow as about 0.1 mm up to about 2 mm, and be spaced at regular intervals such as 180°, 120° or 90 apart. Particularly in these embodiments, the head 11 or upper end of the sheath 16 include an indexing marker, or the like to provide the operator with information as to the orientation of the laser, and particular the irradiating zones.

Prior to using the laser in this protocol, endodontic treatment can be completed by the method of the clinician's choice as long as the protocols utilized fulfill the well-established mechanical and biological objectives required for predictable success. The procedural steps include complete access, followed by negotiating and shaping the canal to facilitate three-dimensional cleaning and obturation of the root canal system. The only unique requirements are threefold: 1) the primary canal must be completely negotiated to its terminal extent; 2) the canal must be prepared into a uniform tapered shape of between 2 and 10% such that each cross-sectional diameter narrows in an apical direction; and 3) the terminal extent of the canal must be minimally enlarged to about 0.20 mm or about 200 microns. This is necessary so that the irradiating fiber tip can reach within about 1 mm of the terminal extent of the preparation. The taper prevents binding and breakage of the exposed fiber in smaller, curved canals. If there is proximity to vital anatomical structures such as the mental foramen or mandibular nerve, an end-capped tip should be selected.

Once a canal has been completely mechanically and chemically prepared, the preparation must be rinsed with EDTA to promote the removal of the smear layer. It should then be rinsed in a sodium hypochlorite solution to neutralize any residual EDTA solution in the canals. The sodium hypochlorite can then be rinsed with sterile saline, sterile water or dried out directly with paper points. In any scenario, excess solutions of any type should be removed with the use of paper points until the paper points are retrieved from the canals consistently dry. Excess water will absorb the laser energy and reduce the available energy available to targeted cells. After these procedural steps have been accomplished, the disposable laser tip is selected and fit so its working end can be inserted to within about 1 mm of the terminal extent of the canal preparation. Importantly, the most coronal extent of the laser's working area must not protrude more than about 1.5 mm into the access cavity to provide protection and prevent lateral radiant laser energy from reaching the clinician, staff, and patient. At this point the procedure depends on which of the two energy phasing embodiments is selected (Such as FIG. 1). In the first, and preferred embodiment, the laser tip releases energy at its tip and laterally simultaneously along the entire length of the working fiber, irradiating the entire canal without the need to move the active tip. In a second embodiment (Such as FIG. 2), the active tip 24 may have zones or bands of laser irradiation and bands where no irradiation may occur. This may be done for purposes of controlling the location of the energy release, reducing the heat distribution to the tooth or to compensate for power levels inadequate to power the active tip effectively. If this embodiment is selected, then the tip will need to be moved, in a coronal direction, until all of the treatment zones have been lased. In either instance, energy release is controlled directly by the laser unit via an automatic shut off. In the instance of irradiating specific zones, then following the completion of laser treatment within any given zone, the energy is automatically shut off signaling the clinician to move to the next band or zone.

To begin the protocol, starting at the root apex, disinfect/sterilize the canal by engaging the power source for the prescribed amount of time, depending upon the embodiment used and move the tip coronally so as not to recontaminate the previously lased area after its sterilization.

A controlled amount of energy is deposited for a particular time at a particular location and distribution within the root canal system. The exact method would depend upon the embodiment selected. If energy application is to be phased, then the tip is to be stepped back coronally in a manner consistent with the use of the calibrations and color-coded markings along the sleeve/sheath or fiber. If the embodiment selected is one in which all of the energy is deposited at once along the entire working length of the fiber and the length of the fiber is long enough to cover the entire length of the canal, then there is no need to proceed in multiple phases. One variation may be the movement of the spiral embodiment once as previously described to treat the areas left untreated by the spiral design and allow the treated areas to cool. Once inserted to the proper depth, the tip is activated for the appropriate amount of time to assure the disinfection of the canal contents along with the ablation/vaporization of the tissue fragments within the primary and secondary anatomy. Once the calculated energy has been deposited, the tip is simply withdrawn and placed in the next primary canal to be treated. When there are multiple canals, this process is repeated for each canal within any given tooth.

After the laser process has been completed for all primary canals, residual charring may be removed by flushing out the canals with solutions of EDTA and sodium hypochlorite. This irrigating process is enhanced by agitating the solution utilizing an instrument manually or via a mechanized way. The canals should then be reflushed with irrigant and dried.

### Optimization of Treatment Energy

The use of a diode laser as an adjunct to the sterilization of the root canal system as described above results in the significant generation of heat in the treated root canal as a byproduct of the laser operation. The ability to keep the heat below biological thresholds that are safe to the surrounding structures, such as nerves, blood vessels, periodontal ligaments and bone is of paramount importance for the safe and effective operation of diode lasers. The more efficiently the delivered energy is used, the less waste heat will be generated.

Irrespective of which embodiment or technique is chosen, the operator may elect to further enhance both the disinfection and cleaning of the canal by subsequently filling the previously treated canal(s) with an aqueous solution and activate the tip again at a low wattage of between 0.5 to 2 watts for short periods of time followed by resting periods to take advantage of the photoacoustic effects of this device.

### Treatment Efficacy and Single Use Design

Another essential ingredient to the successful operation of the diode laser in intracanal endodontic applications, where direct visualization is not possible and work is done “blind”, is some form of system that assures that the full and calculated strength of the radiation is dispensed as prescribed. Degradation of the dispensing tip will result in a reduced level of radiation dose and hence may not accomplish the desired result. Assumption of disinfection when not accomplished is undesirable and may result in treatment failures. Conversely, the turning up of the power to assure disinfection because the operator assumes degradation, but cannot quantify it, is similarly undesirable due to the increased and likely unnecessary extra heat generation and unwanted tissue destruction.

### Safety

Additionally, it is essential to know when the laser tip has extended past the confines and safety of the root proper. Activation of the laser under these conditions below the tooth root and into the gum/tissue area will result in the direct application of laser energy to the surrounding tissues possibly resulting in unintended damage to those tissues.

The inventive embodiments particular to each category listed above are described under their respective headings below. Because of the dramatic effect of the selected wavelength laser operation, and the more efficient in-canal heating targeted to the water contained therein, various tip configurations can enhance the power wave of energy generated by the inventive technique.

### Optimization of Treatment Energy

There are four different inventions/embodiments designed to optimize the use of treatment energy. Treatment energy optimization results in more effective treatment outcomes per unit dose of treatment energy applied. Results related to energy optimization include reducing waste heat needing to be dissipated into the surrounding tissues thereby increasing safety to the surrounding tissues. The rationale, embodiments and methods proposed by this invention to accomplish that result are listed below.

### Reflective Coating(s)

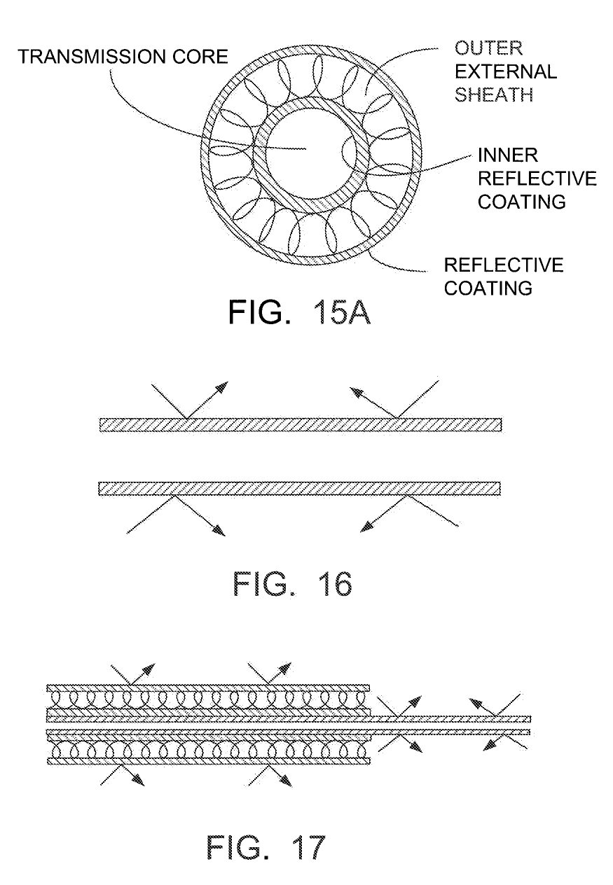

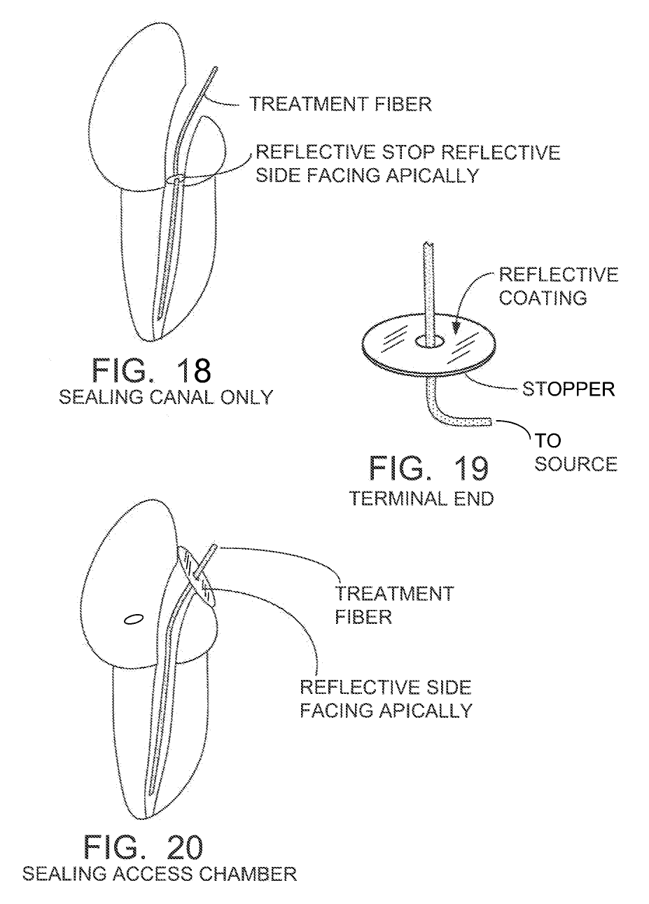

The fiber optic bundle used in endodontic treatment applications is encased in a outermost protective cladding or sheath, hereinafter, “sheath” or “sheathing”. In endodontic treatment applications the protective sheathing may remain intact or be otherwise scored in multiple configurations with the intention of allowing lateral emissions. Such emission angles may vary from one degree to 90 degrees from the long axis of the fiber. The release of treatment energy within the relatively enclosed confines of a root canal system will impact the dentinal walls at different angles resulting in scattering, transmission, absorption and reflection of the treatment energy. The first embodiment is designed to re-reflect the scattered and reflected energies that reach the sheathing material back to the tooth structure as treatment energy. The concept of this embodiment is to coat the outer surface of the sheathing with any reflective coating that will re-reflect energy through multiple iterations until the energy has been ultimately absorbed by the tooth structure or otherwise lost through the coronal aspect of the access to the root canal system. Such a coating is more particularly illustrated in FIG. 15.

### Exposure of the Reflective Surface Underneath the Sheathing

Similar to the application of a reflective coating to the exterior sheathing of the fiber optic bundle as previously described, a variation, and new useful embodiment by the removal of the fiber optic sheathing exposing the optically reflective layer below. The original purpose of the optically reflective coating is to reflect light energy back along the length of the fiber that energy not in the long axis of the fiber which would otherwise be lost in the absence of the optically reflective coating. This is done by using a material in the reflective coating that has a lower index of refraction than does the transmitting core. In this embodiment, some or all of the outermost protective sheathing is removed exposing the external aspect of the optically reflective coating underneath. When exposed to the scattered and reflected treatment energy, the exposed reflective layer below will re-reflect those energies back to the tooth structure as treatment energies. While the reflectivity is not normally as high as an additional reflective coating applied to the outermost sheathing, it can be significant, and its costs sufficiently lower to warrant manufacture.

This exposure of the underlying reflective coating will have the same result as the application of the reflective coating on the exterior surface of the sheathing, i.e. the re-reflection back to the tooth structure of non-absorbed energy and its concomitant results as previously described. This embodiment may be used in combination with the application of a reflective coating applied to the external aspect of the sheathing as described above in that some of the fiber may have the sheathing removed to expose the underlying reflective surface while other areas of the same fiber may be coated with a reflective substance on the external sheathing itself. The combination of both approaches may result in an enhanced treatment result. The two embodiments, one showing the sheathing removal only (FIG. 16) and one showing the combination of sheath removal and sheath reflective coating (FIG. 17) are shown.

### Reflective Stop

Irrespective of whether a reflective coating is exposed or applied to the surface of the external sheathing as previously described, there exists another significant portal of exit for applied treatment energy. That portal is through the occlusal or coronal access to the root canal system, i.e., the entry column of the treating fiber. In principle, it is similar to the insertion of a water hose into a piece of PVC pipe capped on only one end. The water pressure will clean the side walls of the internal aspect of the pipe to a certain extent, but the uncapped and unsealed nature of the pipe at the hose's entrance allows water to exit the pipe reducing the water pressure and its effectiveness inside the pipe itself.

In this embodiment a flexible stop, similar to an endodontic stop as used on endodontic files, and is preferably coated with a reflective material on the side facing the canal opening. Its purpose is to stop the egress of wasted energy in the coronal direction and re-reflect it back into the root canal system as treatment energy. In such an embodiment, the stop will have an appropriate sized hole pre-made through which the treatment fiber 18a is inserted. The combination treatment fiber/reflective stop is then be inserted into the tooth. Once the fiber reaches the prescribed treatment depth, the reflective stop or shield is be slid down the fiber so as to seal either the chamber access or preferably, the entrance to the canal orifice itself. Once so sealed, the treatment energy is dispensed and the reflective stop acts to re-reflect escaping energy back to the treatment zone with the attendant benefits of increased treatment efficacy and waste heat reduction. Examples of this embodiment are illustrated in FIGS. 15, 15A and 17.

### UV Light

UV light is well known to be an effective sterilizing agent. Its application in the sterilization of root canal systems has only recently been explored. While it can be effective in the disinfection of root canal systems, when conventionally applied, it lacks the power to ablate tissue, or penetrate far into the dentinal tubules. Because of this its effects on bacteria embedded in the tubules are uncertain and variable. Despite its efficacy in disinfection, tissue remnants, necrotic and vital, remain intact serving as a future foodstuffs for future bacterial/fungal infections.

The combination of laser energy and UV light in the disinfection of root canal systems has not been commercially explored to date. In the scenario of an effective treatment program, the addition of UV light energy to the root canal system, either before or after the application of laser energy, may result in the ability to use reduced laser energy resulting in less heat to be dissipated by the surrounding tissues resulting again in both greater efficacy and greater safety.

An alternative embodiment incorporates a dual-type emission source in which one source supplies the UV light and run the UV emissions down the treatment fiber then, permitting a switch to the laser emission source and run the laser emissions down the same, or different, fibers. Such a dual source approach offers cost and space efficiencies while allowing for a choice of treatment modalities.

The operator may elect to operate only the UV emissions in areas of delicate anatomy or where the containment of the laser energy cannot be assured. Examples of such areas may include proximate anatomic structures such as the mandibular canal, mental foramen, infraorbital nerve.

### Treatment Efficacy and Single Use Design

Treatment of root canal systems is done “blind” for four primary reasons:
1) The canal space is small and cannot be visualized during treatment.
2) There are many ramifications/accessory canals that extend obliquely from the long axis of the primary canal. The contents of such ramifications/accessory canals cannot be therefore visualized directly.
3) The goal of endodontic treatment is to ablate residual tissue fragments and disinfect/sterilize the primary canals, secondary canals and dentinal tubules. The limits of human vision, and even its augmentation with surgical operating microscopes, do not allow such a level of resolution so as to distinguish individual bacteria, much less their status as living or dead.
4) The insertion of the treatment fiber and the operator's fingers block direct vision at the time of treatment.

Therefore, it is imperative that the treatment tip deliver the amount of treatment energy calculated to be effective in the cleansing/disinfection of the root canal system. It is therefore desirable that a single use application treatment tip be configured so that a consistent, known level of treatment energy can be predictably and reproducibly applied to the root canal system. With repeated use the laser treatment tips suffer breakage of the fiber optic core transmission fibers due to the repeated flexion generated by use in tight and curved root canal systems. Such breakage of fibers disrupts the light throughput reducing the delivered dose of energy and rendering disinfection/sterilization results uncertain. The tips are additionally subject to charring after use—also serving to reduce output during later use. Any indicator, safety, or reflective coatings will be similarly be rendered inactive or unreliable by previous use. For these reasons, it is strongly recommended that the treatment tip should be disposable/single use.

Similarly, current sterilization concerns require such tips should be for single use only. Certain nations have mandated that endodontic files shall be single use only because of the inability of routine sterilization processes in use today to kill prions.

In this embodiment, the tip may also be coated in an indicator that changes color after use or a predetermined amount of use. For example, the disposable tips may be coated green in color as they come from the manufacturer, but turn red after activation with heat, laser or UV energy. Such an indicator should make it easy for the operator and auxiliary staff to distinguish between used and unused tips.

### Safety

In addition to the use of UV light in areas of delicate anatomy, one needs to include provisions for the safe operation of the laser in the advent that such a UV add-on capability is not available in the treating unit.

The calculation of the exact length of the root is more art than science. Hence, it is very easy for the operator to inadvertently extend the treatment laser fiber past the protective confines of the tooth structure itself.

In tooth length determination statistical norms are not adequate in that they are the average of a large population and bear little relevance to the unique, individual, tooth being treated. Failure to compensate for the individual peculiarities at hand can be catastrophic. Angulation of x-rays may produce an image that is longer or shorter than the actual tooth length. Additionally, the end of the root canal confines do not coincide with the radiographic end of the root the majority of times. Electronic apex locators also have mechanical and interpretive error rates that are far from rare and can be fairly significant in degree. Tactile sense alone cannot be relied on due to curves, constrictures, and blockages in the root canal system. Measurement by paper points cannot be relied on exclusively either as there can be bleeding into the canal resulting in a short reading. Another problem with this method of measurement can be the existence of dead space or tissue which is not moist or does not bleed exterior to the confines of the root structure. In practice, the operator will usually rely on more than one modality to make a clinical judgment about the actual tooth length. Such judgments may, or may not, be accurate.

One embodiment used to aid in this process coats the terminal apical end portion of the fiber with a coating that is either water soluble, changes color after exposure to moisture or blood, or uses the precipitation of a char layer after an initial low energy activation to indicate whether or not the proposed laser tip activation zone resides safely within the confines of the root structure. Such change may be induced by dissolving of the primary coating, exposing a different colored undercoating, a chemical reaction induced by the presence of blood or moisture, or the precipitation of a char on the exposed tip surface. Under this embodiment, it is envisioned that such color change/indication shall be rapid enough and visible enough to allow the operator to determine when he/she has exited the confines of the root canal system and is the more vulnerable tissues surrounding the root.

Although the present invention has been described in terms of specific embodiments, it is anticipated that alterations and modifications thereof will no doubt become apparent to those skilled in the art. It is therefore intended that the following claims be interpreted as covering all alterations and modifications that fall within the true spirit and scope of the invention.
^{[1]} Academy of Laser Dentistry. Diode and Nd:YAG lasers clinical case studies. Wavelengths 2004; 12:1.
^{[2]} Bornstein E. Near-infrared dental diode lasers: scientific and photobiologic principles and applications. Dent Today. Mar 2004;23:102-108.
^{[3]} Grant S A, Soufiane A, Shirk G, et. al. Degradation-induced transmission losses in silica optical fibers. Lasers Surg Med. 1997;21:65-71.
^{[4]} Kuhn T S. Black-Body Theory and the Quantum Discontinuity, 1984-1912. Chicago, Ill.: University of Chicago, Press: 1978:1
^{[5]} Manni J G. Dental Applications of Advanced Lasers. Burlington, Mass:JGM Associates, Inc; 2000: Section 1. ^{[6]} Harris D. Dosimetry for laser sulcular debridement. Lasers Surg Med. 2003;33:217-218.
^{[7]} Gregg R H ^{2na,} McCarthy D. Laser periodontal therapy: case reports. Dent Today 2001;20:74-81.
⁸ Gutknecht, N, Schippers R F M, Lampert F, Bactericidal Effect of a 980 nm Diode Laser in the Root Canal Wall Dentin of Bovine teeth.

1. An endodontal laser treatment apparatus including a handpiece, having an elongated laser tip for insertion into the interstices of a tooth, comprising:
a first laser source connected to the handpiece for delivering a laser beam of a predetermined wavelength to the handpiece;
a head connected to the handpiece for receiving the laser beam from the laser source, said head being connected to an optical fiber extending outward of the head, having a core, a cladding surrounding the core and a protective coating around the cladding;
a generally cylindrical laser energy delivery tip terminating said optical fiber, whereby said energy delivery tip is adapted with radiation windows whereby said laser beam may be directed to predetermined endodontal areas of a tooth to be treated.

2. The apparatus of claim 1 wherein said tip has a radiation window disposed in the distal region of the tip for delivery of laser energy generally axially out of the tip.

3. The apparatus of claim 1 wherein said tip has an axial radiation window disposed at the distal end of said tip and a radial window circumferentially up said tip toward said head a predetermined distance whereby the release of laser energy is axial of said tip and radially about the lateral window.

4. The apparatus of claim 1 wherein said tip has a circumferential opening in said cladding and protective cover forming a radial radiation window intermediate the distal end of said tip and said head.

5. The apparatus of claim 5 wherein said tip has a plurality of circumferential radial radiation windows intermediate the distal end of said tip and said head.

6. The apparatus of claim 5 wherein the circumferential window extends around the tip in a helical form from the distal end of the tip for a predetermined distance toward the head.

7. The apparatus of claim 5 wherein there are a plurality of helical windows.

8. The apparatus of claim 1 wherein the protective layer over the optical fiber in the region of the tip includes a depth scale whereby a used of the apparatus may determine the depth to which the distal end of the tip is extended.

9. The apparatus of claim 1 wherein said laser source delivers pulses of laser energy wherein each pulse is for a predetermined duration.

10. The apparatus of claim 9 wherein the laser source delivers pulses in intervals of a predetermined number of pulses.

11. The apparatus of claim 1 wherein the laser source delivers laser energy at a wavelength selected from about 930 nm to about 1065 nm.

12. The apparatus of claim 2 wherein said tip has a radiation window disposed in the distal region of the tip extending axially toward the head for a predetermined distance.

13. The apparatus of claim 12 wherein the tip has a plurality of axial windows disposed around the circumference of the tip.

14. The apparatus of claim 1 wherein the thickness of the cladding on distal tip is tapered beginning at a predetermined distance from the distal tip to whereby the release of radial laser energy increases from zero to a maximum level at the distal tip.

15. The apparatus of claim 1 wherein the laser source is a diode laser.

16. The apparatus of claim 15 wherein the laser delivers energy at a wavelength of about 960 nm to about 1000 nm.

17. The apparatus of claim 16 wherein the laser delivers energy at a wavelength of about 980 nm.

18. The apparatus of claim 1 wherein the diameter of the core, cladding and protective layer are about 200 microns to about 800 microns.

19. The apparatus of claim 1 wherein said tip includes a radial shield disposed at a predetermined distance proximate the distal end of the tip.

20. The apparatus of claim 1 wherein the laser tip includes a protective light stop disposed over the entry to the interstices of the tooth.

21. The apparatus of claim 1 wherein dual light guides provide light of different wavelengths.
